SEQUENCE LISTING (13 sequences)

SEQ ID NO: 1 - protein, 5 aa, Artificial Sequence
  Description of Artificial Sequence Synthetic peptide
VPGXG

SEQ ID NO: 2 - DNA, 327 bp, Artificial Sequence
  Description of Artificial Sequence Synthetic polynucleotide
atgggtgttcaggttgaaaccatctctccgggtgacggtcgtaccttcccgaaacgtggtcagacctgcgttgttcactacaccggtatgctggaagacggtaaaaaattcgactcttctcgtgaccgtaacaaaccgttcaaattcatgctgggtaaacaggaagttatccgtggttgggaagaaggtgttgctcagatgtctgttggtcagcgtgctaaactgaccatctctccggactacgcttacggtgctaccggtcacccgggtatcatcccgccgcacgctaccctggttttcgacgttgaactgctgaaactggaaggt

SEQ ID NO: 3 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 4 - protein, 482 aa, Artificial Sequence
  Description of Artificial Sequence Synthetic polypeptide
GVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 5 - DNA, 1770 bp, Artificial Sequence
  Description of Artificial Sequence Synthetic polynucleotide
atgggtgttcaggttgaaaccatctctccgggtgacggtcgtaccttcccgaaacgtggtcagacctgcgttgttcactacaccggtatgctggaagacggtaaaaaattcgactcttctcgtgaccgtaacaaaccgttcaaattcatgctgggtaaacaggaagttatccgtggttgggaagaaggtgttgctcagatgtctgttggtcagcgtgctaaactgaccatctctccggactacgcttacggtgctaccggtcacccgggtatcatcccgccgcacgctaccctggttttcgacgttgaactgctgaaactggaaggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttccgggttctggtgttccgggctctggtgtaccaggtagcggtgtaccgggttctggcgtacctggctccggtgtcccgggttccggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggtgttcctggtatcggtgttccgggcatcggtgtacctggcattggtgtcccaggtattggcgttccaggtatcggcgtaccaggtattggttac

SEQ ID NO: 6 - protein, 1922 aa, Artificial Sequence
  Description of Artificial Sequence Synthetic polypeptide
GVPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 7 - protein, 15 aa, Artificial Sequence
  Description of Artificial Sequence Synthetic peptide
GGGGSGGGGSGGGGS

SEQ ID NO: 8 - protein, 15 aa, Artificial Sequence
  Description of Artificial Sequence Synthetic peptide
EAAAKEAAAKEAAAK

SEQ ID NO: 9 - protein, 241 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 10 - protein, 2000 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 11 - protein, 5 aa, Artificial Sequence
  Description of Artificial Sequence Synthetic peptide
VPGXG

SEQ ID NO: 12 - protein, 480 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSG

SEQ ID NO: 13 - protein, 240 aa, Artificial Sequence
  Description of Artificial Sequence Synthetic polypeptide
VPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGFGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSG

(19) United States
(12) Patent Application Publication
(10) Pub. No.: US 2015/0209335 A1
(43) Pub. Date: Jul. 30, 2015

(54) METHODS AND SMALL MOLECULE THERAPEUTICS COMPRISING FUSED ELPS

(71) Applicants: John Andrew MacKay, Pasadena, CA (US); Sarah F. Hamm-Alvarez, Pasadena, CA (US); Pu Shi, Alhambra, CA (US); Jugal P. Dhandhukia, Los Angeles, CA (US); Mihir K. Shah, Alhambra, CA (US)

(72) Inventors: John Andrew MacKay, Pasadena, CA (US); Sarah F. Hamm-Alvarez, Pasadena, CA (US); Pu Shi, Alhambra, CA (US); Jugal P. Dhandhukia, Los Angeles, CA (US); Mihir K. Shah, Alhambra, CA (US)

(73) Assignee: UNIVERSITY OF SOUTHERN CALIFORNIA, Los Angeles, CA (US)

(21) Appl. No.: 14/683,033

(22) Filed: Apr. 9, 2015

Related U.S. Application Data
(63) Continuation of application No. PCT/US2013/064719, filed on Oct. 11, 2013.
(60) Provisional application No. 61/713,434, filed on Oct. 12, 2012.

(51) Int. Cl. A61K 31/436; A61K 9/16; C12N 9/90; A61K 47/48; C07K 14/78

(52) U.S. Cl. CPC A61K 31/436; A61K 47/48246; C07K 14/78; C12N 9/90; A61K 47/48853; A61K 9/1658; C12Y 502/01008

(57) ABSTRACT

This disclosure provides a novel compositions and methods to deliver small molecule therapeutics using genetically engineered protein polymers connected to the ‘cognate’ human protein target of that drug.

### CROSS-REFERENCE TO RELATED APPLICATIONS

This application is a continuation of International Application No. PCT/US2013/064719, filed Oct. 11, 2013, which in turn claims priority under 35 U.S.C. §119(e) to U.S. Provisional Application No. 61/713,434, filed Oct. 12, 2012, the entire content of each of which is hereby incorporated by reference into the present disclosure.

### STATEMENT OF GOVERNMENT SUPPORT

This invention was made with government support under Grant No. RO1EY017293-04S1 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 13, 2013, is named 064189-6460_SL.txt and is 81,165 bytes in size.

### BACKGROUND

Synthetic nanoparticles, such as dextran, PLGA, liposomes have been designed as tissue and cell-specific targeting moieties. For example, bilayer phospholipid vesicles decorated with polyethylene glycol (PEG) or coated with charged polymers like poly (acrylic acid) and/or polyallyl amine HCL (PAH) are currently used to encapsulate small molecule drugs. Other known methods include chemically synthesized block co-polymer nanoparticle poly(ethylene glycol)-b-poly((-caprolactone) (PEG-PCL) to encapsulate small molecule drugs such as rapamycin by a co-solvent extraction technique. The nanoparticle performs a slow release with a half-life up to 39 hours. Immunosuppressive small molecule drugs have also been encapsulated in biodegradable polymers like acetylated dextran that forms microparticles following a single-emulsion production technique. The prior art compositions and therapies using them suffer from dose-limiting toxicity, insufficient residence time in the body, and a lack of targeted delivery to intended tissues. This invention overcomes these limitations and provides related advantages as well.

### SUMMARY

This disclosure provides a novel compositions and methods to deliver small molecule therapeutics using genetically engineered protein polymers connected to the ‘cognate’ human protein target of that drug. Most therapeutics, including but not limited to cancer drugs, have dose-limiting toxicity, insufficient residence time in the body, and a lack of targeted delivery to their intended tissues. They may benefit from targeted drug carriers that would carry them specifically to their intended target; however, encapsulation in most drug carriers is achieved through either through chemical bond linkages or through non-specific adsorption or entrapment.

To address this obstacle, the inventors disclose a new, simple concept to use the human protein target for known drugs directly as the drug carrier itself. This encapsulation approach does not rely on either chemistry for attachment or nonspecific physical entrapment, but would instead rely on a high affinity interaction with the very same target that the drug was intended to reach in the body, its ‘cognate’ human receptor. For example, the inventors have evaluated the co-encapsulation of a potent drug called rapamycin in a fusion protein containing human FKBP. The rapamycin binds to the FKBP domain; furthermore, this prevents it from flooding the tissues of the body where side-effects are mediated. Since FKBP does not make an optimal targeted carrier, the inventors fused it to a protein polymeric nanoparticle that provides long-circulation and targeted binding to biomarkers of cancer. This carrier dramatically reduces toxicity for rapamycin, which enables evaluation of this drug as a cancer therapy formulation. This principle can be applied in theory to any small molecule drug with a known human target. The new delivery system reduces dose-limiting toxicity, increases drug bioavailability and increases drug circulation half-life. The problem that is solved is that this approach is a rational strategy that increases the tolerated dose for a wide range of small molecules; furthermore, combinations of fusion protein/drug complexes can be developed into a wide array of highly specific drug carriers.

In one aspect, this disclosure provides an agent comprising, or alternatively consisting essentially of, or yet further consisting of, an elastin-like polypeptide (ELP) component that forms a stable nanoparticle above the transition temperature of the ELP, a ligand and a therapeutic agent. In one aspect, the ELP component is the polypeptide S48I48 (G(VPGSG)n(VPGIG)nY (SEQ ID NO: 6)(wherein n is an integer that denotes the number of repeats, and can be from about 6 to about 192, or alternatively from about 15 to 75, or alternatively from about 40 to 60, or alternatively from about 45 to 55, or alternatively about 48, e.g., S48I48 (G(VPGSG)₄₈(VPGIG)₄₈Y (SEQ ID NO: 4), wherein the integer “48” intends the number of repeats) or a biological equivalent thereof. In one embodiment, the therapeutic agent is trapped within a stable nanoparticle (also known as a “micelle”) formed by the ELP when the environmental temperature is above the transition temperature of the ELP.

A non-limiting example of a therapeutic agent is a small molecule drug. The ligand specifically recognizes and binds the therapeutic agent, i.e., it comprises the cognate target of the therapeutic agent. In one aspect, it is the receptor for the therapeutic agent. Non-limiting examples of agent-ligand pairs include, without limitation rapamycin-FKBP, cyclosporinA-cyclophilin A, Everolimus-FKBP, Temsirolimus-FKBP, Ridaforolimus-FKBP, Tacrolimus-FKBP.

In one aspect, the therapeutic agent is rapamycin and the ligand comprises reference peptide prolyl isomerase protein (also known as reference peptide FK506 binding protein (FKBP)) (SEQ ID NO: 2) or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC and wherein the biological equivalent binds rapamycin.

In other aspects, the therapeutic agent is of the group Everolimus; or Temsirolimus; or Ridaforolimus or Tacrolimus, and the ligand for each comprises reference peptide prolyl isomerase protein (also known as reference peptide FK506 binding protein (FKBP)) (encoded by SEQ ID NO: 2) or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC and wherein the biological equivalent binds the therapeutic agent.

In another aspect, the therapeutic agent is cyclosporin A and the ligand comprises, or alternatively consists essentially of, or yet further consists of cyclophilin A (SEQ ID NO: 3) or a biological equivalent thereof, wherein biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC and binds cyclosporin A.

In each of the above noted aspects, the agent may optionally comprise, or alternatively consist essentially of, or yet further consist of a detectable label.

In each of the above noted aspects, the agent may optionally comprise, or alternatively consist essentially of, or yet further consist of a linker that links the ligand to the therapeutic agent. Non-limiting examples of such include a thiol reactive linker, cleavable disulfide linker, a hydrophilic flexible linker comprised of amino acids (GGGGS)₃ (SEQ ID NO: 7) or a rigid linker comprised of amino acids (EAAAK)₃ (SEQ ID NO: 8), wherein the subscript “3” denotes the number of repeats. In one aspect the peptide can be repeated from 2 to 10, or from 2 to 8, or from 3 to 8, or from 3 to 3 to 5.

Yet further provided is an isolated polynucleotide encoding an elastin-like polypeptide (ELP) component that forms a stable nanoparticle (also known as a micelle) above the transition temperature of the ELP and a ligand that specifically recognizes and binds a cognate target of the agent. The isolated polynucleotide can optionally be operatively linked to regulatory or expression elements that facilitate recombinant expression of the polynucleotide, such as promoters, enhancers, etc. In one aspect, the polynucleotide encodes an ELP component that comprises, or alternatively consists essentially of, or yet further consists of polypeptide S48I48 or a biological equivalent thereof, wherein a biological equivalent of polypeptide S48I48 is a peptide that has at least 80% sequence identity to polypeptide S48I48 or a peptide encoded by a polynucleotide that hybridizes under conditions of high stringency to a polynucleotide that encodes polypeptide S48I48 or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of S48I48.

The polynucleotide also encodes a ligand that is the receptor or ligand of a therapeutic agent. In one aspect, the polynucleotide also encodes reference peptide prolyl isomerase protein (encoded by SEQ ID NO: 2) (also known as peptide FK506 binding protein (FKBP)) or reference peptide cyclophilin A (SEQ ID NO: 3), a biological equivalent of each thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC and optionally operatively linked to expression and/or regulatory sequences. The biological equivalent will also bind the therapeutic agent as it is the cognate target of the agent, e.g., prolyl isomerase protein or FKBP binds rapamycin and the biological equivalent of cyclophilin A binds cyclosporin A.

The polynucleotides can further comprise an expression or replication vector and regulatory sequences for the replication and/or expression of the polynucleotides. In a further aspect, the polynucleotide and/or vector are contained within host cells. The polynucleotides or vectors or host cells can be used to prepare an agent as described herein by expressing the polynucleotide and then in one aspect, isolating the ELP-fusion expressed by the polynucleotide. A composition containing the vector and/or host cell is further provided herein. In one aspect, the polynucleotide sequence encodes FKBP-S48I48 (SEQ ID NO:5) and comprises, or alternatively consists essentially of, or yet further consists of the sequence: ATGGGTGTTCAGGTTGAAACCATCTCTCCGGGTGACGGTCGTACCTTCCCGAA ACGTGGTCAGACCTGCGTTGTTCACTACACCGGTATGCTGGAAGACGGTAAAA AATTCGACTCTTCTCGTGACCGTAACAAACCGTTCAAATTCATGCTGGGTAAAC AGGAAGTTATCCGTGGTTGGGAAGAAGGTGTTGCTCAGATGTCTGTTGGTCAG CGTGCTAAACTGACCATCTCTCCGGACTACGCTTACGGTGCTACCGGTCACCC GGGTATCATCCCGCCGCACGCTACCCTGGTTTTCGACGTTGAACTGCTGAAACT GGAAGGTGTTCCGGGTTCTGGTGTTCCGGGCTCTGGTGTACCAGGTAGCGGTGTACC GGGTTCTGGCGTACCTGGCTCCGGTGTCCCGGGTTCCGGTGTTCCGGGTTCTGGTGTT CCGGGCTCTGGTGTACCAGGTAGCGGTGTACCGGGTTCTGGCGTACCTGGCTCCGGT GTCCCGGGTTCCGGTGTTCCGGGTTCTGGTGTTCCGGGCTCTGGTGTACCAGGTAGC GGTGTACCGGGTTCTGGCGTACCTGGCTCCGGTGTCCCGGGTTCCGGTGTTCCGGGT TCTGGTGTTCCGGGCTCTGGTGTACCAGGTAGCGGTGTACCGGGTTCTGGCGTACCT GGCTCCGGTGTCCCGGGTTCCGGTGTTCCGGGTTCTGGTGTTCCGGGCTCTGGTGTAC CAGGTAGCGGTGTACCGGGTTCTGGCGTACCTGGCTCCGGTGTCCCGGGTTCCGGTG TTCCGGGTTCTGGTGTTCCGGGCTCTGGTGTACCAGGTAGCGGTGTACCGGGTTCTG GCGTACCTGGCTCCGGTGTCCCGGGTTCCGGTGTTCCGGGTTCTGGTGTTCCGGGCTC TGGTGTACCAGGTAGCGGTGTACCGGGTTCTGGCGTACCTGGCTCCGGTGTCCCGGG TTCCGGTGTTCCGGGTTCTGGTGTTCCGGGCTCTGGTGTACCAGGTAGCGGTGTACC GGGTTCTGGCGTACCTGGCTCCGGTGTCCCGGGTTCCGGTGTTCCTGGTATCGGTGTT CCGGGCATCGGTGTACCTGGCATTGGTGTCCCAGGTATTGGCGTTCCAGGTATCGGC GTACCAGGTATTGGTGTTCCTGGTATCGGTGTTCCGGGCATCGGTGTACCTGGCATT GGTGTCCCAGGTATTGGCGTTCCAGGTATCGGCGTACCAGGTATTGGTGTTCCTGGT ATCGGTGTTCCGGGCATCGGTGTACCTGGCATTGGTGTCCCAGGTATTGGCGTTCCA GGTATCGGCGTACCAGGTATTGGTGTTCCTGGTATCGGTGTTCCGGGCATCGGTGTA CCTGGCATTGGTGTCCCAGGTATTGGCGTTCCAGGTATCGGCGTACCAGGTATTGGT GTTCCTGGTATCGGTGTTCCGGGCATCGGTGTACCTGGCATTGGTGTCCCAGGTATT GGCGTTCCAGGTATCGGCGTACCAGGTATTGGTGTTCCTGGTATCGGTGTTCCGGGC ATCGGTGTACCTGGCATTGGTGTCCCAGGTATTGGCGTTCCAGGTATCGGCGTACCA GGTATTGGTGTTCCTGGTATCGGTGTTCCGGGCATCGGTGTACCTGGCATTGGTGTCC CAGGTATTGGCGTTCCAGGTATCGGCGTACCAGGTATTGGTGTTCCTGGTATCGGTG TTCCGGGCATCGGTGTACCTGGCATTGGTGTCCCAGGTATTGGCGTTCCAGGTATCG GCGTACCAGGTATTGGTTAC; wherein, the bolded polynucleotides encodes FKBP (SEQ ID NO: 2), the underlined polynucleotide encodes the ELP and the italicized codon encodes for a tyrosine residue at the carboxy terminus that is not required for activity of the FKBP or ELP domains.

Methods to prepare the agents and ELP-fusions are further provided herein. In one aspect, a method is provided that comprises preparing a composition comprising the therapeutic agent and the ELP-fusion and subsequently raising the environmental temperature of the above the transition temperature of the ELP.

Compositions are further disclosed comprising, or alternatively consisting essentially of, or yet further consisting of a carrier, such as a pharmaceutically acceptable carrier, and one or more of an agent, polynucleotide, expression vector, replication vector, and isolated host cell as described herein and above.

The agents and compositions are useful to deliver a drug in vitro by contacting a tissue with the agent or composition. The agents and compositions also are useful to deliver a drug in vivo by administering an effective amount of the agent or composition as described herein to a subject. In one aspect, the agent or composition is useful for ameliorating the symptoms of a disease or condition or for treating a disease or condition. The method comprises, or alternatively consists essentially of, or yet further consists of, administering an effective amount of the agent or the composition as described herein to a subject suffering from the disease or condition or susceptible to the disease or condition. In one aspect, the disease or condition is cancer.

Kits are also disclosed. The kit is for ameliorating the symptoms of a disease or condition or treating a disease. The kits comprise, or alternatively consist essentially of, or yet further consist of an agent or composition as described herein and instructions for use.

### BRIEF DESCRIPTION OF THE FIGURES

FIG. 1 shows ELP temperature-dependent phase transition. When the temperature is above the transition temperature (Tt), ELP I48, [VPGIG]₄₈Y (SEQ ID NO: 9), phase separates and becomes insoluble in bulk water. When the temperature drops below the transition temperature, ELP reversibly becomes soluble and returns to the solution.

FIG. 2 shows rapamycin (Rapa) encapsulation using F24S24 and 148S48 nanoparticles (also known as micelles). Time 0 h represents the initial encapsulated Rapa after film hydration method. Dialysis analysis was performed after to test the encapsulation stability up to 6 hours. ELP S192 was used as the control because it does not form any nanoparticles. Two-way ANOVA analysis was performed to examine the differences between F24S24/148S48 and S192 groups.

FIG. 3 shows FKBP-S48I48 nanoparticle (also known as a micelle) with rapamycin part of rapamycin is encapsulated inside the nanoparticle (also known as a micelle) core and the rest is bound to FKBP domain.

FIG. 4 shows rapamycin (Rapa) release from ELP nanoparticles with and without FKBP. Dialysis under sink conditions was used track the loss of Rapa from the nanoparticles. Mean±SD (n=3).

FIG. 5 shows MTS cell viability assay using FKBP-S48148 rapamycin and free rapamycin in MDA-MB-468 and MDAMB-231 cell lines.

FIG. 6 shows FKBP-S48I48 rapamycin and free rapamycin tumor regression study in a MDA-MB 468 rapamycin sensitive breast cancer cell line xenografted female athymic nude mouse model FKBP-S48I48 Rapa shows less toxicity and better antitumor activity than free rapamycin.

FIG. 7 shows that FSI-Rapamycin reduces lymphocyte foci in NOD mice. In the top panel, it is shown that after one week of treatment, lacrimal gland histology demonstrated less lymphocyte invasion (dark) in Free Rapamycin and FSI-Rapamycin. The bottom panel is image analysis that was used to quantify % of tissue section containing lymphocyte foci. *p=0.003, **P=5×10·5, ***P=0.01, ****P=0.0009.

FIG. 8 shows that FSI-Rapa reduces interferon gamma in NOD mouse LG. Quantitation was achieved using real time RT-PCR. Expression of IFN-gamma was quantified after a 7-day treatment period with PBS, Free Rapa, and FSI Rapa. There was a sizeable reduction in the cytokine expression levels between the treatment groups and the untreated control. *p=0.02.

FIG. 9 shows that FKBP nanoparticles reduce toxicity of Rapamycin in NOD mice. In the top panel, mice were administered Rapa (40% EtOH), Vehicle (40% EtOH), or FSI-Rapa anoparticles via tail vein injections. Images and body weights are tabulated 7 days after first, (2 days after last) administration. FSI significantly reduces body weight loss *p=0.0006.

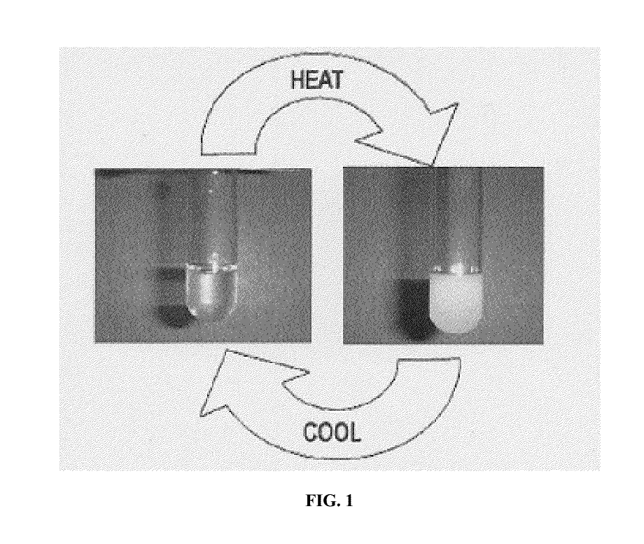

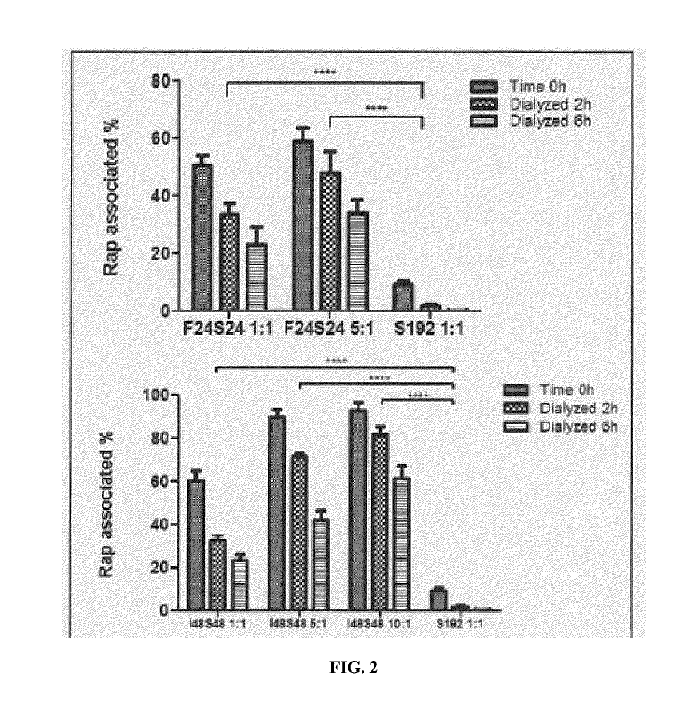

### PARTIAL DESCRIPTION OF THE SEQUENCES

SEQ ID NO:1 is the amino acid sequence of the pentapeptide (VPGXG)n wherein n is an integer representing the number of repeats and X denotes any amino acid.

SEQ ID NO:2 is the polynucleotide sequence encoding the amino acid (polypeptide) sequence of FKBP.

SEQ ID NO:3 is the amino acid (polypeptide) sequence of cyclophilin A.

SEQ ID NO:4 is the amino acid sequence of S48I48.

SEQ ID NO:5 is the polynucleotide sequence of a vector expressing the S48I48 fused to FKBP.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. In the context of this application, the active agent is the ELP-containing a ligand and therapeutic agent as described herein. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

The term “therapeutic” refers to an agent or component capable of inducing a biological effect in vivo and/or in vitro. The biological effect may be useful for treating and/or preventing a condition, disorder, or disease in a subject or patient. A therapeutic may include, without limitation, a small molecule, a nucleic acid, or a polypeptide. Non-limiting examples of such include rapamycin and cyclosporin A.

As used herein, the term “elastin-like peptide (ELP) component” intends a polypeptide that forms stable nanoparticle (also known as a micelle) above the transition temperature of the ELP. In one aspect, the ELP component comprises, or alternatively consists essentially of, or yet further consists of the polypeptide S48I48 having the sequence G(VPGSG)n(VPGIG)nY (SEQ ID NO: 6) (wherein n is an integer that denotes the number of repeats, and can be from about 6 to about 192, or alternatively from about 15 to 75, or alternatively from about 40 to 60, or alternatively from about 45 to 55, or alternatively about 48), wherein in one aspect, S48I48 comprises, or alternatively consists essentially of, or yet further consists of the amino acid sequence G(VPGSG)₄₈(VPGIG)₄₈Y (SEQ ID NO: 4), or a biological equivalent thereof. A biological equivalent of polypeptide S48I48 is a peptide that has at least 80% sequence identity to polypeptide S48I48 or a peptide encoded by a polynucleotide that hybridizes under conditions of high stringency to a polynucleotide that encodes polypeptide S48I48 or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of S48I48.

Rapamycin is a small molecule drug with the IUPAC name (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29 (4H,6H,31H)-pentoneis). It is an immunosuppressant drug used to prevent rejection in organ transplantation and has been used in the treatment of cancers. It is marketed under the trade name Rapamune™ by Pfizer.

The mammalian target of rapamycin is known as mTOR or FK506 binding protein 12-rapamycin associated protein 1 (FRAP1, referenced herein is FK506 Binding Protein or “FKBP”), is a protein that in humans is encoded by the FRAP1 gene. The protein and gene sequence encoding the protein are disclosed under GenBank Accession No. NG_{—}033239 (last accessed on Sep. 6, 2013). mTOR is a serine/threonine protein kinase that regulates cell growth, proliferation, cell survival, protein synthesis among other functions.

Cyclosporin A is a small molecule immunosuppressant drug widely used in organ transplants and has been successfully used in the treatment of cardiac disease. The IUPAC name for the drug is (3S,6S,9S,12R,15S,18S,21S,24S,30S,33S)-30-Ethyl-33-[(1R,2R,4E)-1-hydroxy-2-methyl-4-hexen-1-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone. It is sold under the trade names Neoral™ or Sandimmune™. It binds the cytosolic protein cyclosporine A.

Cyclophilin A is also known as peptidylprolyl isomerase A. It is found in the cytosol. The sequence of the human protein and polynucleotide encoding the protein is disclosed under GenBank Accession No.: NP_{—}066953 (last accessed on Oct. 7, 2013). A published amino acid sequence comprises MVNPTVFFDI AVDGEPLGRV SFELFADKVP KTAENFRALS TGEKGFGYKG SCFHRIIPGF MCQGGDFTRH NGTGGKSIYG EKFEDENFIL KHTGPGILSM ANAGPNTNGS QFFICTAKTE WLDGKHVVFG KVKEGMNIVE AMERFGSRNG KTSKKITIAD CGQLE (SEQ ID NO.: 3).

Everolimus is the 40-O-(2-hydroxyethyl) derivative of sirolimus and works similarly to sirolimus as an inhibitor of the mammalian target of rapaycin. It is marketed under the tradenames Zortress (USA) and Certican (Europe and other countries) in transplantation medicine, and Afinitor in oncology. Everolimus also is available with Biocon with the brand name of Evertor. It is used as an immunosuppresent to prevent rejection of organ transplants and the treatment of tumors such as renal cell cancer. The compound also is known as dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone.

Temsirolimus is (CCI-779) is a derivative of sirolimus and is sold as Torisel. It is an intravenous drug for the treatment of renal cell carcinoma, developed by Wyeth Pharmaceuticals. It also is approved by the European Medicines Agency (EMEA) on November 2007. The compound also is known as (1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate.

Ridaforolimus (also known as AP23573 and MK-8669; formerly known as Deforolimus) is an investigational targeted and small-molecule inhibitor of the protein mTOR. The compound also is known as (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4.9}]hexatriaconta-16,24,26,28-tetraen-12-yl]propyl]-2-methoxycyclohexyl dimethylphosphinate.

Tacrolimus (also FK-506 or fujimycin, trade names Prograf, Advagraf, Protopic) is an immunosuppressive drug that is mainly used after allogeneic organ transplant to reduce patient rejection. The drug also is known as 3S[3R*[E(1S*,3S*,4S*)],4S*,5R*,8S*,9E,12R*,14R*,15S*,16R*,18S*,19S*,26aR*5,6,8,11,12,13,14,15,16,17,18,19,24,25,26,26a-hexadecahydro-5,19-dihydroxy-3-[2-(4-hydroxy-3methoxycyclohexyl)-1-methylethenyl]-14,16-dimethoxy-4,10,12,18-tetramethyl-8-(2-propenyl)-15,19-epoxy-3H-pyrido[2,1-c] [1,4]oxaazacyclotricosine-1,7,20,21(4H,23H)-tetrone, monohydrate.

As used herein, the term “biological equivalent thereof” is used synonymously with “equivalent” unless otherwise specifically intended. When referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide or with respect to polynucleotides, those hybridize under stringent conditions to the reference polynucleotide or its complement. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

An “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as known to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, topical application, intraperitoneal, intravenous and by inhalation. An agent of the present invention can be administered for therapy by any suitable route of administration. It will also be appreciated that the preferred route will vary with the condition and age of the recipient, and the disease being treated.

The agents and compositions of the present invention can be used in the manufacture of medicaments and for the treatment of humans and other animals by administration in accordance with conventional procedures, such as an active ingredient in pharmaceutical compositions.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine. In terms of cells, the term “mammalian cells” includes, but is not limited to cells of the following origin: a human, a feline, a canine, a simian, a murine, a bovine, an equine, a porcine or an ovine.

As used herein, the term “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

### Elastin-Like Polypeptides (ELPs)

Elastin-like-polypeptides (ELPs) are a genetically engineered polypeptide with unique p hase behavior (see for e.g. S. R. MacEwan, et al., Biopolymers 94(1) (2010) 60-77), which promotes recombinant expression, protein purification, and self-assembly of nanostructures (see for e.g. A. Chilkoti, et al., Advanced Drug Delivery Reviews 54 (2002) 1093-1111). ELPs are artificial polypeptides composed of repeated pentapeptide sequences, (Val-Pro-Gly-Xaa-Gly)n (SEQ ID NO: 1) derived from human tropoelastin, where Xaa is the “guest residue” which is any amino acid, an amino acid analog or amino acid derivative thereof. In one embodiment, Xaa is any amino acid except proline. This peptide motif displays rapid and reversible de-mixing from aqueous solutions above a transition temperature, Tₜ. Below Tₜ, ELPs adopt a highly water soluble random coil conformation; however, above Tₜ, they separate from solution, coalescing into a second aqueous phase. The Tₜ of ELPs can be tuned by choosing the guest residue and ELP chain length as well as fusion peptides at the design level (see for e.g. MacEwan S R, et al., Biopolymers 94(1): 60-77). The ELP phase is both biocompatible and highly specific for ELPs or ELP fusion proteins, even in complex biological mixtures. Genetically engineered ELPs are monodisperse, biodegradable, non-toxic. Throughout this description, ELPs are identified by the single letter amino acid code of the guest residue followed by the number of repeat units, n. For example, S48I48 represents a diblock copolymer ELP with 48 serine (S) pentamers at the amino terminus and 48 isoleucine (I) pentamers at the carboxy terminus.

Described herein are ELP fusion proteins, which can be self-assembled into nanoparticles (alternatively known as micelles). The diameter of the nanoparticle can be from about 1 to about 1000 nm or from about 1 to about 500 nm, or from about 1 to about 100 nm, or from about 1 to about 50 nm, or from about 20 to about 50 nm, or from about 30 to about 50 nm, or from about 35 to about 45 nm. In one embodiment, the diameter is about 40 nm. These nanoparticles can be high efficiently internalized, e.g. into LGAC. The fusion proteins are composed of elastin-like-polypeptides and high affinity polypeptides. These fusion proteins can be expressed from a variety of expression systems known to those skilled in the art and easily purified by the phase transition behavior of ELPs. These ELP fusion proteins are able to conjugate small molecules, such as, for example, chemotherapeutic agents, anti-inflammation agents, antibiotics and polypeptides and other water soluble drugs. In addition, the ELP nanoparticles are useful for carrying DNA, RNA, protein and peptide-based therapeutics.

ELPs have potential advantages over chemically synthesized polymers as drug delivery agents. First, because they are biosynthesized from a genetically encoded template, ELPs can be made with precise molecular weight. Chemical synthesis of long linear polymers does not typically produce an exact length, but instead a range of lengths. Consequently, fractions containing both small and large polymers yield mixed pharmacokinetics and biodistribution. Second, ELP biosynthesis produces very complex amino acid sequences with nearly perfect reproducibility. This enables very precise selection of the location of drug attachment. Thus drug can be selectively placed on the corona, buried in the core, or dispersed equally throughout the polymer. Third, ELP can self-assemble into multivalent nanoparticles that can have excellent site-specific accumulation and drug carrying properties. Fourth, because ELP are designed from native amino acid sequences found extensively in the human body they are biodegradable, biocompatible, and tolerated by the immune system. Fifth, ELPs undergo an inverse phase transition temperature, Tₜ, above which they phase separate into large aggregates. By localized heating, additional ELP can be drawn into the target site, which may be beneficial for increasing drug concentrations.

As disclosed herein, the ELPs of this disclosure are attached to a receptor that binds to therapeutic small-molecule ligands. Non-limiting examples of such include FKBP that is the ligand for rapamycin or cyclophilin which is the ligand for cyclosporin A. The ELP and receptor are fused directly through a covalent peptide linkage, which is genetically encoded at the level of the DNA.

A therapeutic such as a drug, for example, may be attached to the ELP through cysteine, lysine, glutamic acid or aspartic acid residues present in the polymer. In some embodiments, the cysteine, lysine, glutamic acid or aspartic acid residues are generally present throughout the length of the polymer. In some embodiments, the cysteine, lysine, glutamic acid or aspartic acid residues are clustered at the end of the polymer. In some embodiments of the presently described subject matter, therapeutics are attached to the cysteine residues of the ELP using thiol reactive linkers. In some embodiments of the presently described subject matter, therapeutics are attached to the lysine residues of the high molecular weight polymer sequence using NHS (N-hydroxysuccinimide) chemistry to modify the primary amine group present on these residues. In some embodiments of the presently described subject matter, therapeutics are attached to the glutamic acid or aspartic acid residues of the ELP using EDC (1-Ethyl-3-[3-dimethylaminopropyl]carbodiimide Hydrochloride) chemistry to modify the carboxylic acid group present on the ELP residues.

The therapeutic associated with the ELP may be hydrophobic or hydrophilic. Which the drug is hydrophobic, attachment to the terminus of the ELP may facilitate formation of the multivalent nanoparticle. The number of drug particles attached to the ELP can be from about 1 to about 30, or from about 1 to about 10, or about 1, 2, 3, 4, 5, 6, 7, 8, 9, or 10. In some embodiments, the attachment points for a therapeutic are equally distributed along the backbone of the ELP, and the resulting drug-ELP is prevented from forming nanoparticle structures under physiological salt and temperature conditions.

In addition to therapeutics, the ELPs may also be associated with a detectable label that allows for the visual detection of in vivo uptake of the ELPs. Suitable labels include, for example, fluorescein, rhodamine, tetramethylrhodamine, eosin, erythrosin, coumarin, methyl-coumarins, pyrene, Malacite green, Alexa-Fluor®, stilbene, Lucifer Yellow, Cascade Blue.™., and Texas Red. Other suitable optical dyes are described in Haugland, Richard P. (1996) Molecular Probes Handbook.

In certain embodiments, the ELP components include polymeric or oligomeric repeats of the pentapeptide (VPGXG)n (SEQ ID NO: 10), wherein n is an integer representing the number of repeats between 5 and 400, alternatively between 5 and 300, or alternatively between 25 and 250, or alternatively between 25 and 150, and wherein the guest residue X (also denoted as Xaa herein) is any amino acid, that in one aspect, excludes proline. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine.

The guest residue X may be a non-classical (non-genetically encoded) amino acid. Examples of non-classical amino acids include: D-isomers of the common amino acids, 2,4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The ELP component in some embodiments is selected or designed to provide a Tₜ ranging from about 10 to about 80° C., such as from about 35 to about 60° C., or from about 38 to about 45° C. In some embodiments, the Tₜ is greater than about 40° C. or greater than about 42° C., or greater than about 45° C., or greater than about 50° C. The transition temperature, in some embodiments, is above the body temperature of the subject or patient (e.g., >37° C.) thereby remaining soluble in vivo, or in other embodiments, the Tₜ is below the body temperature (e.g., <37° C.) to provide alternative advantages, such as in vivo formation of a drug depot for sustained release of the therapeutic agent.

The Tₜ of the ELP component can be modified by varying ELP chain length, as the Tt generally increases with decreasing MW. For polypeptides having a molecular weight >100,000, the hydrophobicity scale developed by Urry et al. (PCT/US96/05186, which is hereby incorporated by reference in its entirety) is preferred for predicting the approximate Tₜ of a specific ELP sequence. However, in some embodiments, ELP component length can be kept relatively small, while maintaining a target Tₜ, by incorporating a larger fraction of hydrophobic guest residues (e.g., amino acid residues having hydrophobic side chains) in the ELP sequence. For polypeptides having a molecular weight <100,000, the Tₜ may be predicted or determined by the following quadratic function: Tₜ=M₀+M₁X+M₂X² where X is the MW of the fusion protein, and M₀=116.21; M₁=−1.7499; M₂=0.010349.

While the Tₜ of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, and half-life of the molecule.

### Expression of Recombinant Proteins

ELPs and other recombinant proteins described herein can be prepared by expressing polynucleotides encoding the polypeptide sequences of this invention in an appropriate host cell, i.e., a prokaryotic or eukaryotic host cell This can be accomplished by methods of recombinant DNA technology known to those skilled in the art. It is known to those skilled in the art that modifications can be made to any peptide to provide it with altered properties. Polypeptides of the invention can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that beta-turn spiral secondary structure or random secondary structure is preferred.

The ELPs can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells includes Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Protein Purification

The phase transition behavior of the ELPs allows for easy purification. The ELPs may also be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “[n]-fold purification number” wherein “n” is an integer. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxyapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Pharmaceutical Compositions

Pharmaceutical compositions are further provided. The compositions comprise a carrier and an agent, an ELP-fusion with a ligand, or a polynucleotide encoding the ELP-fusion, as described herein or other compositions (e.g., polynucleotide, vector system, host cell) as described herein. The carriers can be one or more of a solid support or a pharmaceutically acceptable carrier. In one aspect, the compositions are formulated with one or more pharmaceutically acceptable excipients, diluents, carriers and/or adjuvants. In addition, embodiments of the compositions include ELPs, formulated with one or more pharmaceutically acceptable auxiliary substances.

The invention provides pharmaceutical formulations in which the one or more of an agent, ELP-fusion with a ligand, or a polynucleotide, vector or host cells can be formulated into preparations for injection or other appropriate route of administration in accordance with the invention by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents.

Aerosol formulations provided by the invention can be administered via inhalation. For example, embodiments of the pharmaceutical formulations of the invention comprise a compound of the invention formulated into pressurized acceptable propellants such as dichlorodifluoromethane, propane, nitrogen and the like.

Embodiments of the pharmaceutical formulations of the invention include those in which the composition is formulated in an injectable composition. Injectable pharmaceutical formulations of the invention are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the invention.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Routes of administration applicable to the methods and compositions described herein include intranasal, intraperitoneal, intramuscular, subcutaneous, intradermal, topical application, intravenous, nasal, oral, inhalation, intralacrimal, retrolacrimal perfusion along the duct, intralacrimal, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In one embodiment, the composition comprising the ELP and agent is administered intralacrimally through injection. In further embodiments, the composition is administered systemically, topically on top of the eye, by retrolacrimal perfusion, or intranasally.

### Treatment of Disease

In one aspect, this disclosure provides methods and compositions useful in treating cancer, e.g., breast cancer. As is apparent to those of skill in the art, the cancer to be treated will vary with the therapeutic agent encapsulated and the ligand of the ELP. Non-limiting examples of additional disorders can include, age-related macular degeneration, Sjögren's syndrome, autoimmune exocrinopathy, diabetic retinopathy, graft versus host disease (exocrinopathy associated with) retinal venous occlusions, retinal arterial occlusion, macular edema, postoperative inflammation, uveitis retinitis, proliferative vitreoretinopathy and glaucoma. In one embodiment, the disease is Sjögren's syndrome. In another embodiment, the disease is keratoconjunctivitis sicca (dry eye). In another embodiment the disease is scleritis. In another embodiment the disease is glaucoma.

### Use of Compounds for Preparing Medicaments

The ELPs of the present disclosure are also useful in the preparation of medicaments to treat a variety of pathologies as described herein. The methods and techniques for preparing medicaments of a composition are known in the art. For the purpose of illustration only, pharmaceutical formulations and routes of delivery are detailed herein. In one aspect when the ELP is combined with another therapy or therapeutic agent, provided herein the compositions are useful in the preparation of combination compositions that can be simultaneously or concurrently administered.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Kits

The ELPs as described herein, can be provided in kits. The kits can further contain additional therapeutics and optionally, instructions for making or using the ELPs. In a further aspect, the kit contains reagents and instructions to perform a screen as detailed herein.

### Screening Assays

This invention also provides screening assays to identify potential therapeutic agents of known and new compounds and combinations. For example, one of skill in the art can also determine if the ELP provides a therapeutic benefit in vitro by contacting the ELP or combination comprising the ELP with a sample cell or tissue to be treated. The cell or tissue can be from any species, e.g., simian, canine, bovine, ovine, rat, mouse or human.

The contacting can also be performed in vivo in an appropriate animal model or human patient. When performed in vitro, the ELPs can be directly added to the cell culture medium. When practiced in vitro, the method can be used to screen for novel combination therapies, formulations or treatment regimens, prior to administration to an animal or a human patient.

In another aspect, the assay requires contacting a first sample comprising suitable cells or tissue (“control sample”) with an effective amount of an ELP as disclosed herein and contacting a second sample of the suitable cells or tissue (“test sample”) with the ELP, agent or combination to be assayed. In one aspect in the case of cancer, the inhibition of growth of the first and second cell samples are determined. If the inhibition of growth of the second sample is substantially the same or greater than the first sample, then the agent is a potential drug for therapy. In one aspect, substantially the same or greater inhibition of growth of the cells is a difference of less than about 1%, or alternatively less than about 5% or alternatively less than about 10% , or alternatively greater than about 10% , or alternatively greater than about 20%, or alternatively greater than about 50%, or alternatively greater than about 90%. The contacting can be in vitro or in vivo. Means for determining the inhibition of growth of the cells are well known in the art.

In a further aspect, the test agent is contacted with a third sample of cells or tissue comprising normal counterpart cells or tissue to the control and test samples and selecting agents that treat the second sample of cells or tissue but does not adversely affect the third sample. For the purpose of the assays described herein, a suitable cell or tissue is described herein such as cancer or other diseases as described herein. Examples of such include, but are not limited to cancer cell or tissue obtained by biopsy, blood, breast cells, colon cells.

Efficacy of the test composition is determined using methods known in the art which include, but are not limited to cell viability assays or apoptosis evaluation.

In yet a further aspect, the assay requires at least two cell types, the first being a suitable control cell.

The assays also are useful to predict whether a subject will be suitably treated by this invention by delivering an ELP to a sample containing the cell to be treated and assaying for treatment, which will vary with the pathology, or for screening for new drugs and combinations. In one aspect, the cell or tissue is obtained from the subject or patient by biopsy. This disclosure also provides kits for determining whether a pathological cell or a patient will be suitably treated by this therapy by providing at least one composition of this invention and instructions for use.

The test cells can be grown in small multi-well plates and is used to detect the biological activity of test compounds. For the purposes of this invention, the successful ELP or other agent will block the growth or kill the cancer cell but leave the control cell type unharmed.

### Combination Treatments

Administration of the therapeutic agent or substance of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

As is apparent to those skilled in the art, the combination therapy can take the form of a combined therapy for concurrent or sequential administration.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### EXAMPLES

### 1. Biosynthesis of ELPs

Derived from human tropoelastin, elastin-like polypeptides (ELPs) are amino acid pentamers with the sequence of (Val-Pro-Gly-Xaa-Gly)n (SEQ ID NO: 11), where Xaa is the guest residue that can be any amino acid except proline, and n is an integer and represents the number of the repetitive units. ELPs have a unique feature of phase separation, whereby they undergo temperature-dependent self-assembly. Below a tunable transition temperature (Tt), these ELPs are highly soluble; however, above Tt they can self-associate. Inverse transition cycling (ITC) is utilized to purify ELP samples by taking advantages of this unique feature. A hot centrifugation is performed after triggering ELP phase transition (above Tt) after which ELP pellet can be obtained. The pellet is then re-solubilized in fresh cold PBS followed by a cold centrifugation in order to remove insoluble proteins and contaminations. The cycling is then repeated 4-6 times to obtain pure ELP samples. The purity and peptide MW can be examined by protein SDSPAGE and MALDI-TOF mass spectrometry.

A library of recombinant pET25b+ vectors containing ELP DNA fragments was expressed in BLR E-coli cells. ELP diblock copolymer S48I48 represents the amino acid sequence G(VPGSG)₄₈(VPGIG)₄₈Y (SEQ ID NO: 4). A DNA sequence encoding FKBP was inserted at N-terminus of S48I48 sequence using standard molecular cloning technique. Both S48I48 and FKBP-S48I48 assemble nanoparticle nanoparticles above a critical nanoparticle temperature (CMT), which is associated with phase separation of the isoleucine-containing blocks. ELP block copolymers aggregate above a bulk transition temperature (Tt) dependent upon the serine-containing blocks. Both ELPs settle down to the pellet under heated centrifugation (T>Tt). Under cold centrifugation, ELP remain soluble enabling removal of insoluble protein contaminants. The purity and peptide MW were examined by protein SDS-PAGE and MALDI-TOF mass spectrometry.

### 2. Encapsulation and Release of Rapamycin Using ELP Nanoparticles

A two phase method was developed to encapsulate Rapa into the ELP nanoparticles. An aqueous phase PBS containing S48I48 or FKBP-S48I48 was mixed with an organic phase hexane/EtOH containing Rapa in a small glass vial. The vial was kept stirred and heated up to the CMT of S48I48 or FKBP-S48I48. A nitrogen flow was applied to facilitate the evaporation of the hexane/EtOH phase. A 13.2K 10 min centrifugation was performed to remove the insoluble Rapa after the organic phase evaporated out. 100 μL of the sample was filtered and injected into a C-18 reverse phase HPLC column to analyze the amount of the Rapa that was initially encapsulated. Rapa releasing experiment was performed under a sink condition of H₂O dialysis. Samples were collected in the dialysis cassette at time points of 0 h, 1 h, 2 h, 4 h, 6 h, 9 h, 19.5 h, 27 h, 31 h and 48 h. RP-HPLC was used to determine the amount of Rapa that was retained inside the ELP nanoparticle core.

### 3. ELP Nanoparticles and EM Imaging

ELP diblock copolymers with hydrophobic and hydrophilic guest residues at opposite ends of the polymer (e.g. (Val-Pro-Gly-Ile-Gly) 48(Val-Pro-Gly-Ser-Gly)48 (SEQ ID NO: 12) called I48S48) can form nanoparticles. A class of ELP block copolymers with hydrophobic block:hydrophilic block=1:1 have been confirmed to form different sizes of stable nanoparticles. For instances, (Val-Pro-Gly-Ile-Gly)48(Val-Pro-Gly-Ser-Gly)48 (SEQ ID NO: 12) short name: 148S48; (Val-Pro-Gly- Phe-Gly)24(Val-Pro-Gly-Ser-Gly)24 (SEQ ID NO: 13)) short name F24S24. Dynamic light scattering (DLS) can be applied to measure the size of ELP nanoparticles. At concentration of 25 uM, ELP block copolymer 148S48 forms 24 nm in radius nanoparticles; however, F24S24 forms nanoparticles with 15 nm hydrodynamic radius. In order to actually observe the morphology of ELP nanoparticles, transmission electron microscopy (TEM) and cryo-transmission electron microscopy (Cryo-TEM) can be applied. In TEM, 1% of Uranyl acetate solution is used to negatively stain ELP nanoparticles. Therefore, ELP nanoparticles are expected to be observed as hollow spheres. Different from TEM in which ELP samples are loaded on a grid and dry at room temperature. Cryo-TEM studies ELP samples at cryogenic temperatures, generally liquid nitrogen temperatures. Therefore, particles may be observed without drying-down step and may avoid the formation of ELP aggregates. Because there is no staining in Cryo-TEM, the nanoparticle size will be smaller than TEM as the hydrophilic part of the nanoparticle cannot be observed in Cryo-TEM.

### 4. Rationale of Drug Encapsulation Using ELP Nanoparticles and Advantages

When the temperature is below ELP Tt, ELP single molecules are the main population in solution. However, when the temperature goes above Tt, the hydrophobic blocks start aggregate to forma nanoparticle core while the hydrophilic blocks are sticking outside forming the nanoparticle corona. Some hydrophobic small molecules such as the hydrophobic drug rapamycin can be entrapped into nanoparticle core via hydrophobic interactions and hydrogen bond interactions. Because ELPs are neutrally charged, there is no ionic interactions because ELP and small molecules. The inventors assume that there are two prerequisites for small molecules to be entrapped into ELP nanoparticles. 1) High Log P value; 2) Large number of HBA and/or HBD. A small molecule which satisfy prerequisite 1) or 2) or both 1) and 2) will be efficiently encapsulated into ELP nanoparticle core. ELP nanoparticle encapsulation of drugs has a number of its advantages. Compared to plain drugs, drugs that are encapsulated into ELP nanoparticles will potentially have lower cytotoxicity because of less in vivo exposure. Many cancer therapeutics have limited bioavailability because of their poor water solubility. By entrapping into hydrophobic environment of nanoparticle core, these drugs can reach much higher dose and achieve better bioavailability. Nanoparticle encapsulation effectively shields these drugs from being recognized and eliminated by immune system and therefore prolongs the circulation half-life of these drugs.

### 5. Introduction of Rapamycin and FKBP, Nonspecific Encapsulation and Specific Encapsulation

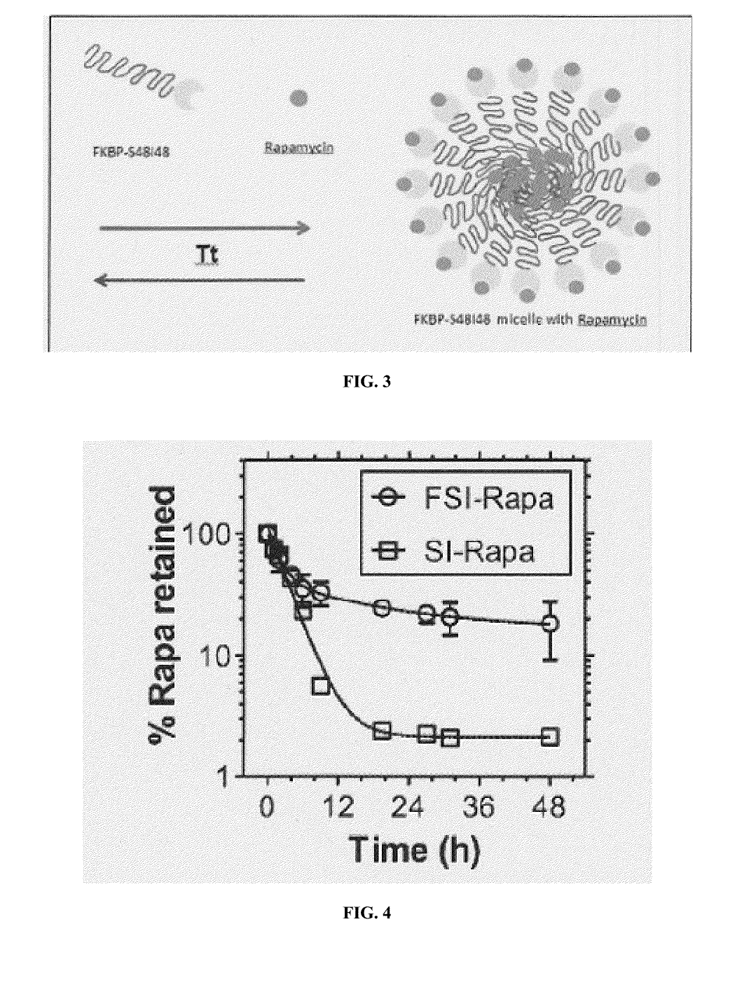

FK506 binding protein (FKBP) is a family of prolyl isomerase proteins and well known for binding immunosuppressant tacrolimus and sirolimus. Rapamycin, another name of sirolimus forms a complex with FKBP and triggers mTOR-Akt pathways in mammalian cells. The rapamycin-FKBP complex is very stable with a Kd of 0.2 nM. Recently, rapamycin has been widely tested on the treatment of different cancer models, and a couple of studies have entered clinical trials. Because of Its large cyclic hydrophobic backbone, rapamycin is poorly dissolved in H₂O (less than 10 μM). Therefore, the bioavailability of the plain drug is very low (less than 20%). However, because rapamycin satisfies both prerequisite of ELP nanoparticle encapsulation 1) high Log P value and 2) large number of HBA and/or HBD, the inventors assumed that rapamycin may have high association with ELP nanoparticles. Preliminary experiments proved that rapamycin could be efficiently encapsulated into plain ELP nanoparticles I48S48 and F24S24. However, because the weak strength of noncovalent hydrophobic and hydrogen bond interactions, the half-life of rapamycin encapsulation of plain ELPs is about 2 hours. To improve rapamycin encapsulation half-time, FKBP domain has been genetically fused onto the hydrophilic block terminus of ELP nanoparticles 848148. Because of FKBP fusion, two different populations of rapamycin can be associated with FKBP-S48I48 nanoparticles: rapamycin that is encapsulated inside the nanoparticle core and rapamycin that is bound to FKBP. (FIG. 3). Preliminary data demonstrated that during rapamycin release experiment, the part of rapamycin that is entrapped into nanoparticle core releases much faster than that is bound to FKBP domain. The encapsulated rapamycin (about 70%) maintains releasing half-time about 2 hrs; however, rapamycin bound to FKBP (about 30%) has a releasing half-time of 58 hrs. (FIG. 4) As a result, FKBP fusion significantly increases rapamycin releasing half-life. Furthermore, taking FKBP-rapamycin as an example, by genetically fusing specific drug-binding domains onto the corona of ELP nanoparticles, a novel drug-specific encapsulation and delivery can be realized.

6. FKBP-S48I48 Rapamycin Nanoparticles have Anti-Tumor Activity

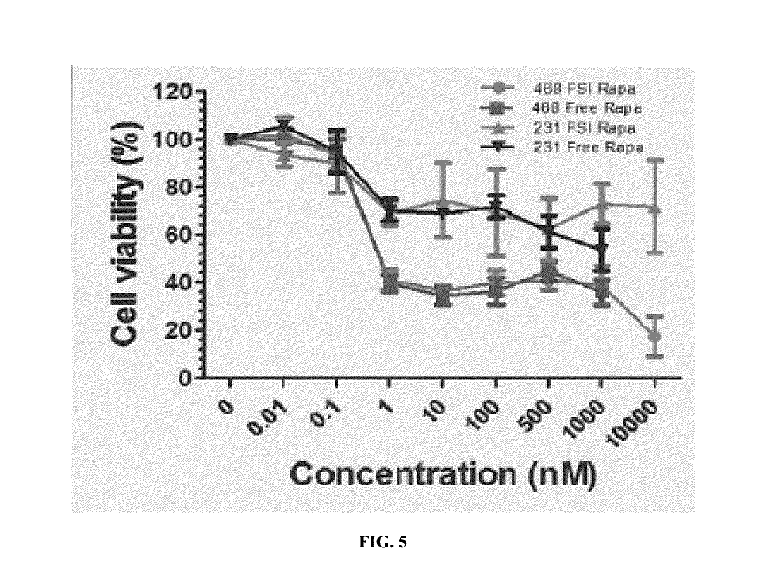

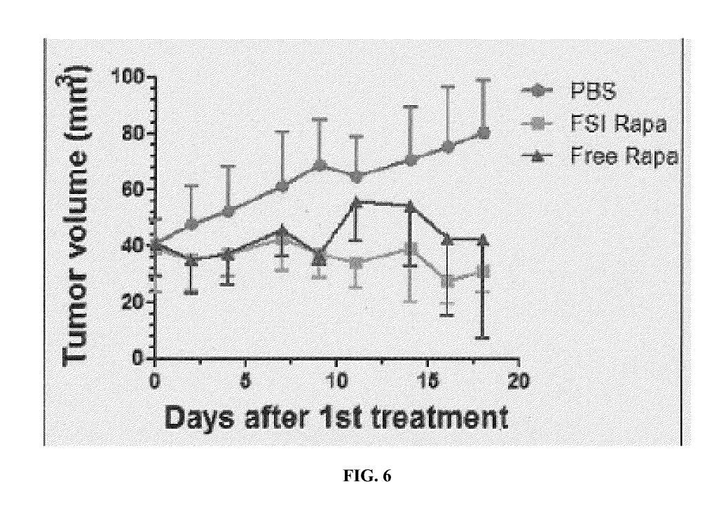

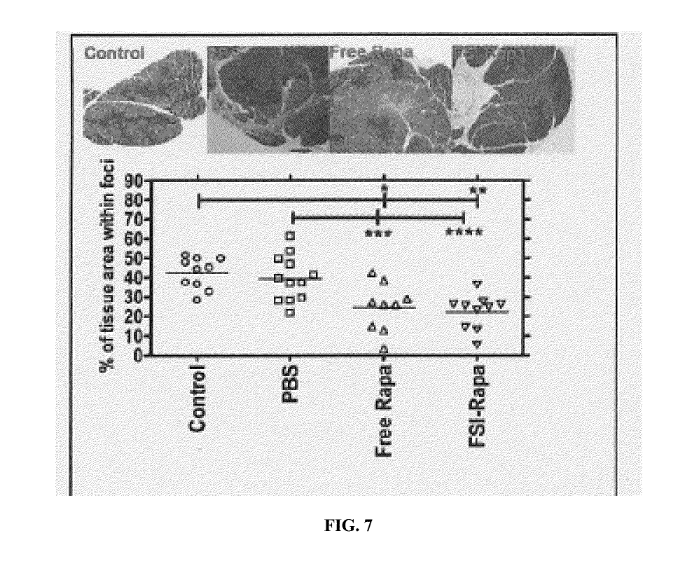

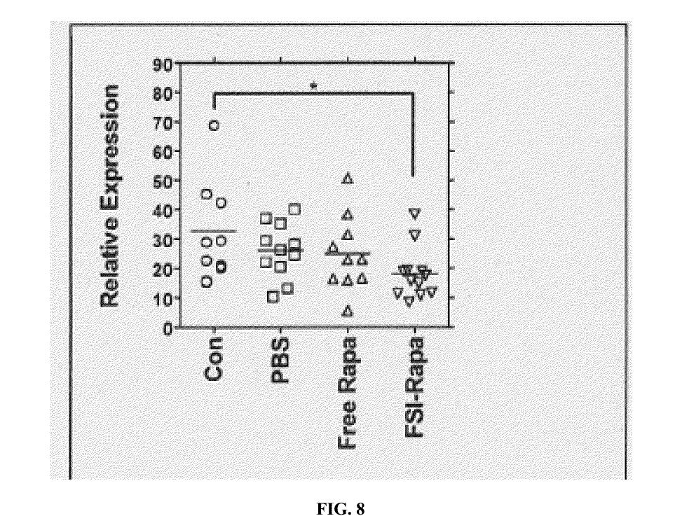

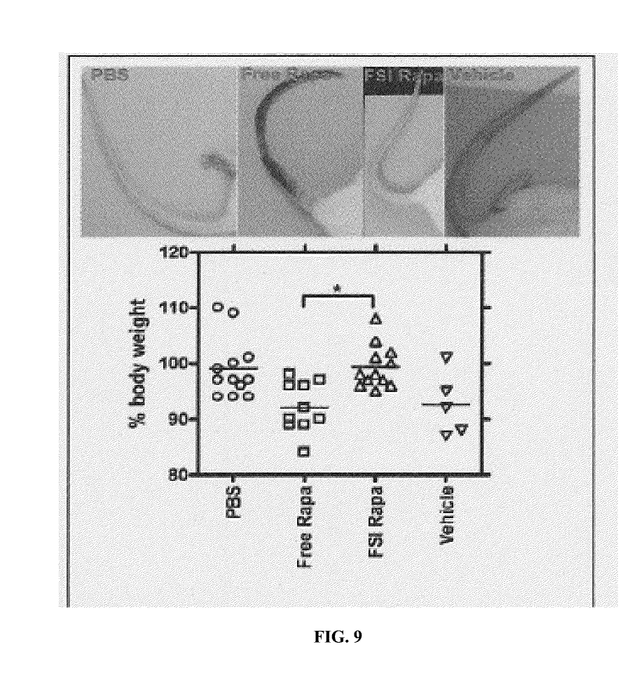

Preliminary experiments examined anti-tumor activity of rapamycin encapsulated FKBP-S48I48 nanoparticles and free rapamycin in MDA-MB-468 rapamycin sensitive breast cancer cell line and MDA-MB-231 rapamycin insensitive breast cancer cell line. It has been demonstrated that in MDA-MB-468 cell, FKBP-S48I48 rapamycin could reach a rapamycin dose of 10 μM and killed −85% of the cells; however, free rapamycin could not reach such a high rapamycin concentration. (FIG. 5) On the contrary, in MDA-MB-231 cell, neither FKBP-S48I48 rapamycin nor free rapamycin could achieve more than 50% cell killing even at very high rapamycin concentrations. The result proves that the rapamycin that is encapsulated in FKBPS48I48 ELP nanoparticles is as potent as free rapamycin; however, ELP nanoparticles can increase rapamycin concentration at least 10 folds to about 10 μM. FKBP-S48I48 rapamycin has been further tested in a MDAMB-468 rapamycin sensitive breast cancer cell line xenografted female athymic nude mouse model. Each mouse was injected with 3×106 MDA-MB-468 cells s.c. in mammary fat pads. 11 days after implantation, 100 μl 150 uM rapamycin FKBP-S48I48 and the same volume of PBS were injected Lv. 3 times a week. Free rapa was initially dosed 3 times a week; however, after first 2 doses, free rapa mice lost nearly 10% of body weight. Therefore, the dose was reduced to only once a week. After that, mice in free Rapa group started to gain weight. The preliminary data shows that tumor size of PBS group is statistically larger than FKBP- S48I48 Rapa group (p=0.007) and free Rapa group (p=0.010). (FIG. 6) FKBP-S48I48 rapamycin shows less toxicity and better anti-tumor activity for MDA-MB-468 breast cancer cells than free rapamycin in vivo. As part of Applicants' preliminary demonstration of efficacy, FSI-Rapamycin was administered via tail vein into 12 week old male NOD mice, an autoimmune mouse model of autoimmune dacryoadenitis. By this age, these mice develop significant lymphocytic infiltration of the lacrimal gland that is a classic characteristic of the aforementioned disease. The results show that FSI-Rapa had a greater therapeutic effect relative to a same amount of the free drug as evidenced by a significant minimization in the lymphocytic infiltration (FIG. 7). The efficacy of the construct was also assessed by measuring changes in a disease associated cytokine, interferon gamma (IFN gamma) that is expressed in the lacrimal gland and observed that treatment with FSI-Rapa led to a more significant reduction in this cytokine as compared to the free drug (FIG. 8). The inventors also observed that this treatment was associated with reduced toxicity and weight loss as can be seen by no necrotic damage of the tail tissue when compared with the free drug (FIG. 9).

The development of an ELP based nanocarrier fused with cyclophilin A can also be developed. The human cognate target for cyclosporin A (CsA) that is a potent immunosuppressant with significant potential for treatment of dacryoadenitis, which is associated with autoimmune disease. As observed with FSI-Rapa, this approach is designed to solve problems of low solubility and toxicity associated with CsA thus reducing the dose-limiting toxicity and help in significant remission of dacryoadenitis and treatment of autoimmune diseases.

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. An agent comprising an elastin-like peptide (ELP) component that forms a stable nanoparticle above the transition temperature of the ELP, a therapeutic agent and a ligand that is the target or receptor of the therapeutic agent.

2. The agent of claim 1, wherein the therapeutic agent is trapped within a stable nanoparticle formed by the ELP when the ELP is above the transition temperature of the ELP.

3. The agent of claim 1, further comprising a linker between the ligand and the ELP.

4. The agent of claim 3, wherein the linker is a thiol reactive linker.

5. The agent of claim 1, wherein the therapeutic agent is an anticancer agent or therapeutic.

6. The therapeutic agent of claim 1, wherein the therapeutic agent is rapamycin and the ligand comprises FK506 binding protein (FKBP) (encoded by SEQ ID NO: 2), or a biological equivalent thereof, wherein a biological equivalent of the FKBP protein is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and binds rapamycin.

7. The agent of claim 1, wherein the therapeutic agent is cyclosporin A and the ligand comprises cyclophilin A (SEQ ID NO: 3) or a biological equivalent thereof, wherein biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and binds cyclosporin A.

8. The agent of claim 1, further comprising a detectable label.

9. The agent of claim 1, wherein the ELP comprises reference polypeptide S48I48 (G(VPGSG)n(VPGIG)nY (SEQ ID NO: 6)(wherein n is an integer that denotes the number of repeats, and can be from about 6 to about 192, or alternatively from about 15 to 75, or alternatively from about 40 to 60, or alternatively from about 45 to 55, or alternatively about 48) or a biological equivalent thereof, wherein a biological equivalent of S48I48 is a peptide that has at least 80% sequence identity to S48I48 or a peptide encoded by a polynucleotide that hybridizes under conditions of high stringency to a polynucleotide that encodes S48I48 or its complement, wherein conditions of high stringency comprise hybridization reaction at about 60° C. in about 1×SSC.

10. A composition comprising the agent of claim 1 and a carrier.

11. The composition of claim 10, wherein the carrier is a pharmaceutically acceptable carrier.

12. A method for delivering a therapeutic agent in vitro comprising contacting a tissue with the agent of claim 1.

13. A method for delivering a drug in vivo comprising administering an effective amount of the agent of claim 1 to a subject.

14. A method for ameliorating the symptoms of a disease or condition or for treating a disease or condition, comprising administering an effective amount of the agent of claim 1 to a subject suffering from the disease or condition or susceptible to the disease or condition.

15. The method of claim 14, wherein the disease or condition is cancer.

16. A kit for ameliorating the symptoms of a disease or condition or treating a disease, comprising an agent of claim 1 and instructions for use.

17. An isolated polynucleotide encoding an elastin-like peptide (ELP) component that forms a stable nanoparticle above the transition temperature of the ELP and a ligand that is a target or a receptor of a therapeutic agent.

18. The isolated polynucleotide of claim 17, wherein the ELP comprises reference polypeptide S48I48 or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC.

19. The isolated polynucleotide of claim 17, wherein the ligand comprises polypeptide (FKBP) or a biological equivalent thereof, wherein a biological equivalent of FKBP is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and optionally operatively linked to expression and/or regulatory sequences.

20. The isolated polynucleotide of claim 17, wherein ligand comprises cyclophilin A or a biological equivalent thereof, wherein a biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and optionally operatively linked to expression and/or regulatory sequences.

21. An isolated vector or isolated host cell comprising an isolated polynucleotide of any one of claims 17 to 20.

22. A composition comprising one or more of the polynucleotide of any one of claims 17 to 20 and a carrier.

23. A method for preparing an ELP-fusion, comprising expressing the polynucleotide of claim 17.

24. The method of claim 23, further comprising isolating the ELP-fusion expressed by the polynucleotide.

25. The method of claim 24, further comprising preparing a composition comprising the therapeutic agent and the ELP-fusion and subsequently raising the temperature of the above the transition temperature of the ELP.

26. A method for preparing the agent of claim 1, comprising preparing a composition comprising the therapeutic agent and the ELP component and subsequently raising the temperature of the composition above the transition temperature of the ELP.
